(19) United States
(12) Patent Application Publication
(10) Pub. No.: US 2014/0170099 A1
(43) Pub. Date: Jun. 19, 2014

(54) REMOVING TYPE AQUEOUS MANICURE COMPOSITION

(71) Applicant: MITSUBISHI PENCIL COMPANY, LIMITED, Shinagawa-ku (JP)

(72) Inventors: Satoshi SAKUMA, Fujioka-shi (JP); Mitsuo Azuma, Yao-shi (JP); Yosuke Ikemoto, Yao-shi (JP)

(73) Assignee: MITSUBISHI PENCIL COMPANY, LIMITED, Shinagawa-ku (JP)

(21) Appl. No.: 14/107,091

(22) Filed: Dec. 16, 2013

(30) Foreign Application Priority Data
Dec. 18, 2012 (JP) 2012-276172

(51) Int. Cl. A61K 8/81; A61Q 3/02

(52) U.S. Cl. CPC A61K 8/8147; A61Q 3/02
USPC 424/61

(57) ABSTRACT

Provided is a removing type aqueous manicure composition which is mild to nails while staying thereon and which can readily be removed by hot water and commercial soaps without using removers containing acetone and acetate esters, so that it can meet a volatile organic compound (VOC) emission control.

The above removing type aqueous manicure composition comprises at least water, an emulsion of an acrylic base resin which has a glass transition temperature (Tg) of 50° C. or higher and which is alkali-soluble, a film-forming aid, and a pH modifier of non-volatile at ambient temperature.

This Nonprovisional application claims priority under 35 U.S.C. §119(a) on Patent Application No. 2012-276172 filed in Japan on 18 Dec. 2012, the entire contents of which are hereby incorporated by reference.

### BACKGROUND OF THE INVENTION

1. Field of the Invention

The present invention relates to a removing type aqueous manicure composition, more specifically to a removing type aqueous manicure composition which can readily be removed by hot water and soaps without using a nail remover containing such as acetone.

2. Description of Related Art

A large variety of blend compositions have so far been developed as aqueous manicure compositions. In general, nitrocellulose and acetates are not used in aqueous manicure compositions, and therefore they are characterized by that they are mild to nails and do not damage the nails.

However, nail removers have to be used when removing manicures, and involved therein is the problem that components such as acetone are contained in the removers and result in damaging the nails.

Known as conventional aqueous manicure compositions are, for example, aqueous manicure compositions which are prepared by using acrylic base polymer emulsions having a glass transition temperature (Tg) of −10 to 90° C. and which are not removed in bathing (refer to, for example, patent document 1), and oil-based compositions for makeup which are prepared by using block copolymers having a glass transition temperature (Tg) of 20° C. or higher and which are excellent in an abrasion resistance (refer to, for example, patent document 2).

Also, known are aqueous cosmetics for a nail art containing alkali-soluble acrylic resins, titanium oxide, acid dyes or organic pigments, and water (refer to, for example, patent documents 3 and 4 each filed by the present Applicant).

However, the manicure compositions disclosed in patent documents 1 and 2 are highly water resistant, and commercial nail removers have to be used for removing the manicures, so that the nails are likely to be damaged to some extent.

The aqueous cosmetics disclosed in patent documents 3 and 4 are used for drawing patterns on a base nail and different from the present invention in a technical basis to a large extent. That is, a film-forming aid is not comprised in the above component constitutions, and therefore when a resin having a high glass transition temperature (Tg) is used therefor, even films are not obtained even if they are applied on the whole parts of the nails. Also, when a resin having a low glass transition temperature (Tg) is used, the problem that the sufficiently hard films are not obtained is involved therein. It is described therein that in order to solve the above problems, the films are finished by applying topcoats in a final stage, and they are apparently different from the present invention in a technical basis to a large extent.
Patent document 1: Japanese Patent Application Laid-Open No. 2003-26540 (Claims, Examples, and others)
Patent document 2: Japanese Patent Application Laid-Open No.

2005-510598 (Claims, Examples, and others)
Patent document 3: Japanese Patent Application Laid-Open No. 2011-140449 (Claims, Examples, and others)
Patent document 4: Japanese Patent Application Laid-Open No. 2011-140462 (Claims, Examples, and others)

### SUMMARY OF THE INVENTION

In light of the foregoing problems and existing situations of the conventional arts, the present invention intends to solve them, and an object thereof is to provide a removing type aqueous manicure composition which can readily be removed by hot water and soaps without using a nail remover damaging nails.

In light of the foregoing problems of the conventional arts, the present inventors intend to solve them and have found that a removing type aqueous manicure composition which meets the object described above is obtained by containing at least water, a resin emulsion having specific physical properties, a film-forming aid, and a specific pH modifier, and thus they have come to be complete the present invention.

That is, the present invention resides in the following items (1) to (3).
(1) A removing type aqueous manicure composition comprising at least water, an emulsion of an acrylic base resin which has a glass transition temperature (Tg) of 50° C. or higher and which is alkali-soluble, a film-forming aid, and a pH modifier of non-volatile at ambient temperature.
(2) The removing type aqueous manicure composition as described in the above item (1), further comprising a pH modifier of volatile at ambient temperature.
(3) The removing type aqueous manicure composition as described in the above item (1), comprising the emulsion of the alkali-soluble acrylic base resin having an acid value of 40 mg KOH/g or more.

According to the present invention, provided is a removing type aqueous manicure composition which can readily be removed by hot water and soaps without using a remover and therefore does not damage nails and which does not cause troubles in a daily life since stable to cold water and can be used easily by children and aged persons.

### DETAILED DESCRIPTION OF THE PREFERRED EMBODIMENTS

The embodiments of the present invention shall be explained below in detail.

The removing type aqueous manicure composition of the present invention is characterized by comprising at least water, an emulsion of an acrylic base resin which has a glass transition temperature (Tg) of 50° C. or higher and which is alkali-soluble, a film-forming aid, and a pH modifier of non-volatile at ambient temperature.

The acrylic base resin of the acrylic base resin emulsion used in the present invention has a glass transition temperature (Tg) of 50° C. or higher and is alkali-soluble. The above acrylic base resin of the emulsion is a resin having no film-forming property at ambient temperature (25° C.; hereinafter the same shall apply), but this resin forms an even coating film at ambient temperature by virtue of the film-forming aid described later.

The acrylic base resin of the emulsion having a glass transition temperature (Tg) of 50° C. or higher, preferably 60° C. or higher is preferably used in terms of forming a crystallized hard coating film after drying and providing the water proof film (water containing no soap) at ambient temperature. The resin having a glass transition temperature (Tg) of 60° C. or higher is preferably used. An upper limit of Tg shall not specifically be restricted, but if Tg exceeds 100° C., an adhesive property of the resin to nails is poor, and therefore Tg is preferably 100° C. or lower. If Tg is lower than 50° C., the resin is insufficiently hard, and therefore it is not preferred.

The glass transition temperature (Tg) was measured in the following manner. First, an acrylic base resin which was turned into a lacquer was coated, and then the solvent was dried up, whereby the coated resin was turned into a film. The film was used to measure a differential scanning calorie by means of a differential scanning calorimeter (DSC) manufactured by Rigaku Corporation, wherein the temperature was elevated from −20° C. up to +150° C. at a heating rate of 10° C/minute. An inflection point was determined to be as Tg by differentiating the obtained endothermic curve.

The acrylic base resin of the acrylic base resin emulsion has to be alkali-soluble.

The acrylic base resin having an acid value of 40 mg KOH/g or more, more preferably 40 to 150 mg KOH/g is preferred from the viewpoint of a balance between durability against cold water and removability by hot water. In the present invention, the acid value is a mg number of KOH necessary to neutralize carboxyl groups contained in 1 g of a resin, and it means a value obtained by a petroleum product and lubricant-neutralizing test method (a potentiometric titration method) according to JIS K2501:2003.

If the above solid acid value is less than 40 mg KOH/g, the water resistance grows too high, and the coating film which cannot be removed even by using soaps and hot water is formed in a certain case.

The acrylic base resin emulsion which can be used includes such as alkyl acrylate copolymer emulsions, acrylate/methacrylate copolymer emulsions, and alkyl acrylate/styrene copolymer emulsions, which meet the properties described above, and the alkyl acrylate/styrene copolymer emulsions are preferably used in terms of a hardness and a glossiness of the film.

A content of the above acrylic base resin emulsions having the above properties is 10 to 40% by mass (hereinafter referred to merely as “%”), preferably 15 to 35% in terms of a solid concentration based on a total amount of the manicure composition.

If a content of the above acrylic base resin of the emulsion is less than 10%, the coating film is thin, and the sufficiently thick film is not obtained. On the other hand, if it exceeds 40%, the emulsion is too viscose to result in obtaining no smoothness on a surface of the coating film.

In the present invention, the effects of the present invention are obtained by using emulsion type alkali-soluble resins. Such the emulsion type alkali-soluble resins can be gradually dissolved from the surfaces thereof by exposed to hot water thereon and can be removed like a thin film, and therefore it is important that they are an emulsion. Also, if they are turned into an emulsion at a lower pH, they are very advantageously blended in a higher concentration.

The film-forming aid used in the present invention shall not specifically be restricted as long as it has a function which enables the acrylic base resin having the properties described above to form an even coating film at ambient temperature. Especially, glycol ethers having a high boiling point are available and include, for example, at least one selected from such as diethylene glycol dimethyl ether, dipropylene glycol dimethyl ether, diethylene glycol ethyl methyl ether, diethylene glycol diethyl ether, dipropylene glycol monomethyl ether, diethylene glycol monomethyl ether, diethylene glycol monobutyl ether, and dipropylene glycol monobutyl ether.

Among the above glycol ethers, preferred are the glycol ethers having a boiling point of 150° C. or higher and solubility of 30 wt % or more, preferably 50 wt % or more into water at 25° C.

Diethylene glycol diethyl ether and dipropylene glycol monobutyl ether are preferably used from the viewpoint of a safety to human bodies.

A content of the above film-forming aids is 5 to 20%, preferably 6 to 15% based on a total amount of the manicure composition.

If a content of the above film-forming aids is less than 5%, a film-forming property of the resin at ambient temperature is not obtained, and therefore it is not preferred. On the other hand, if it exceeds 20%, the film is reduced in a water resistance and a hardness, and therefore it is not preferred.

The pH modifier used in the present invention is added for accelerating removal of the manicure by hot water, and since it stays in an interface between an inside of the coating film and the nail even after drying the coating film, at least the pH modifier of non-volatile at ambient temperature has to be used.

For example, such as aminomethylpropanol, triethanolamine, diethanolamine, sodium hydroxide, and calcium hydroxide can be used as the pH modifier of non-volatile at ambient temperature.

Further, a pH modifier of volatile at ambient temperature, for example, aqueous ammonia is preferably used in combination with the pH modifier of volatile described above from the viewpoint of a cold water resistance and the like of the film.

A content of the above pH modifier is controlled so that a pH of the manicure composition falls in a range of 6 or more and 9 or less, preferably 6.5 to 8.5. If the pH is less than 6, the film is not excellent in a removing property. On the other hand, if it exceeds 9, the film is provided with a skin irritation and deteriorated in a cold water resistance, and it is reduced as well in a drying resistance.

The removing type aqueous manicure composition of the present invention is regulated in a balance by water such as refined water, distilled water, ion-exchanged water, and purified water, and optional components such as a coloring material, a water-soluble organic solvent, a fungicide, a defoamer, and viscosity modifier in addition to the respective components described above can suitably be added as long as the effects of the present invention are not damaged.

Pigments, dyes and the like can be listed as the coloring material available to the present invention.

The available pigments shall not specifically be restricted as long as they are usually used as pigments for liquid cosmetics, and inorganic pigments and organic pigments are available.

It includes, for example, at least one selected from inorganic pigments such as carbon blacks, black iron oxide, yellow iron oxide, chromium oxide, ultramarine, Prussian blue, zinc oxide, aluminum oxide, silica, magnesium oxide, chromium hydroxide, calcium carbonate, titan yellow, and red iron oxide, pearl pigments (highly brilliant particles) such as titanium dioxide-coated scaly titanium, red iron oxide-coated mica titanium, fish scale guanines, and N-ε-lauroyl-L-lysine-coated talc, and organic pigments such as Blue No. 1 Al lake, Red No. 220, Red No. 226, Red No. 228, Blue No. 201, Blue No. 204, Blue No. 404, Yellow No. 401, Yellow No. 205, Yellow No. 4 Al lake, Yellow No. 203 Al lake, and Red No. 104 Al lake, and the like.

A dispersant, if necessary, when the pigments described above are used is selected according to a compatibility with the pigments used, and acrylic base resins and surfactants are used therefor and shall not specifically be restricted.

The available dyes shall not specifically be restricted as long as they are usually used as dyes for liquid cosmetics.

The above dyes are preferably, for example, dyes which have so far been used for cosmetics and the like and which are safe enough to human bodies, and they include, for example, nitro dyes, azo dyes, nitroso dyes, triphenylmethane dyes, xanthene dyes, quinoline dyes, anthraquinone dyes, indigo dyes, and the like. To be specific, capable of being listed from the viewpoint of the dyeing power are, for example, Red No. 2, Red No. 3 (FD & C Red No. 3), Red No. 40 (FD & C Red No. 40), Red No. 102, Red No. 104 (D & C Red No. 28), Red No. 105, Red No. 106, Red No. 201 (D & C Red No. 6), Red No. 202 (D & C Red No. 7), Red No. 203, Red No. 205, Red No. 227 (D & C Red No. 33), Red No. 230-1 (D & C Red No. 22), Red No. 401, Red No. 402, Red No. 504 (FD & C Red No. 4), Orange No. 205 (D & C Orange No. 4), Orange No. 402, Yellow No. 4 (FD & C Yellow No. 5), Yellow No. 5 (FD & C Yellow No. 6), Yellow No. 203 (D & C Yellow No. 10), Yellow No. 402, Yellow No. 403-1 (Ext. D & C Yellow No. 7), Yellow No. 406, Yellow No. 407, Green No. 3 (FD & C Green No. 3), Green No. 201, Green No. 402, Blue No. 1 (FD & C Blue No. 1), Blue No. 2 (FD & C Blue No. 2), Blue No. 203, Blue No. 205 (D & C Blue No. 4), Blue No. 403, Blue No. 404, Brown No. 201 (D & C Brown No. 1), Violet No. 401 (Ext. D & C Violet No. 2), Black No. 401, and the like. At least one selected from the above dyes are available and shall not specifically be restricted as long as a dye available to liquid cosmetics.

When the dye is used, a UV absorber is available, if necessary, in a suitable amount in order to inhibit the fading caused by light.

The UV absorber includes, for example, at least one selected from benzophenone derivatives such as 2-hydroxy-4-methoxybenzophenone, 2-hydroxy-4-methoxybenzophenone-5-sulfonic acid, sodium 2-hydroxy-4-methoxybenzophenone-5-sulfonate, dihydroxymethoxybenzophenone, sodium dihydroxymethoxybenzophenonesulfonate, 2,4-dihydroxybenzophenone, and tetrahydroxybenzophenone, paraaminobenzoic acid derivatives such as paraaminobenzoic acid, ethyl paraaminobenzoate, glyceryl paraaminobenzoate, amyl paradimethylaminobenzoate, and octyl paradimethylaminobenzoate, methoxycinnamic acid derivatives such as ethyl paramethoxycinnamate, isopropyl paramethoxycinnamate, octyl paramethoxycinnamate, 2-ethoxyethyl paramethoxycinnamate, sodium paramethoxycinnamate, potassium paramethoxycinnamate, and glyceryl paramethoxycinnamate mono-2-ethylhexanoate, salicylic acid derivatives such as octyl salicylate, phenyl salicylate, homomenthyl salicylate, propylene glycol salicylate, ethylene glycol salicylate, myristyl salicylate, and methyl salicylate, urocanic acid, ethyl urocanate, 4-tert-butyl-4′-methoxybenzoylmethane, 2-(2′-hydroxy-5′-methylphenyl)bonzoyltriazole, methyl anthranilate, fine particle titanium oxide, fine particle zinc oxide, and fine particle zirconium oxide.

A total content of the coloring materials such as above pigments and dyes is preferably 0.001 to 20%, more preferably 0.01 to 10% based on a total amount of the manicure composition from the viewpoints of obtaining a color nail composition and enhancing the ageing stability, the coating performance, the high glossiness, and the like.

The removing type aqueous manicure composition of the present invention is prepared by mixing and dispersing the respective components described above at contents falling in the respective ranges described above by means of a mix disperser, for example, a bead mill, a homomixer, a disperser, an attritor, and the like.

The removing type aqueous manicure composition of the present invention is suitably available in the forms of, for example, a manicure applicator comprising a bottle (vessel) in which the above manicure composition is stored and a brush which is a coating member mounted in a cap, and a manicure applicator comprising at least a liquid storing space in which the removing type aqueous manicure composition is stored and a coating member of a so-called pen feed type prepared by fixing fibers with a resin and the like or fusing the fibers, wherein the liquid is transported from the liquid storing space to the coating member by virtue of a capillary force.

The removing type aqueous manicure composition of the present invention comprises at least water, the emulsion of the acrylic base resin which has a glass transition temperature of 50° C. or higher and which is alkali-soluble, the film-forming aid, and the pH modifier of non-volatile at ambient temperature, and the above aqueous manicure composition thus constituted is applied on a nail, whereby the acrylic resin which does not form a film at ambient temperature is turned into a film by the action of the film-forming aid and turned in turn into a hard coating film, and the beautiful manicure is obtained. Further, the acrylic resin used has a high Tg, and therefore the coating film after dried is hard and has a high glossiness. The film can be removed by hot water or soaps (commercially available solid soaps, liquid soaps and the like) without using commercially available removers, and therefore the nails are not damaged. On the other hand, the film is stable to cold water (water of 15° C. or lower), and therefore obtained is the removing type aqueous manicure composition which does not cause troubles in a daily life and which can be used easily by children and aged persons.

### EXAMPLES

The present invention shall be explained in further details with reference to examples and comparative examples, but the present invention shall not be restricted by the following examples.

### Examples 1 to 5 and Comparative Examples 1 to 5

The components were mixed and dispersed in the compositions shown in the following Table 1 by means of a homomixer or a disperser to prepare the respective manicure compositions.

The respective manicure compositions obtained in Examples 1 to 5 and Comparative Examples 1 to 5 described above were used to evaluate a hardness, a cold water resistance and removability by the following methods. The results thereof are shown in the following Table 1.

### Measuring Method of pH:

A pH at 25° C. of the respective manicure compositions was measured by means of a pH meter, F-14, manufactured by HORIBA, Ltd.

### Evaluating Method of Hardness:

The respective manicure compositions obtained above were coated on the nails of both hands and left standing for 15 minutes after coated to dry them, and the dried coating films were scratched thereon lightly with a nail and evaluated according to the following evaluation criteria:
◯: not scratched
Δ: scratched a little
×: scraped

### Evaluating Method of Cold Water Resistance:

The respective manicure compositions obtained above were coated on the nails of both hands and left standing for 15 minutes after coated to dry them, and the dried coating films were dipped in cold water (15° C.) for 15 seconds and then rubbed thereon lightly with a finger to evaluate them according to the following evaluation criteria:
◯: no change
Δ: whitened a little on a surface
×: removed

### Evaluating Method of Removability:

The respective manicure compositions obtained above were coated on the nails of both hands and left standing for 15 minutes after coated to dry them, and the dried coating films on the nails were rubbed well with a soap (usual hand soap). Then, the films were rubbed with a finger while causing tepid water to flow to evaluate them according to the following evaluation criteria:
◯: removed
Δ: partially removed
×: not removed or dissolved on the surface and not removed

TABLE 1

| | | Example | | | | | Comparative Example | | | | |
|---|---|---|---|---|---|---|---|---|---|---|---|
| Blend components | | 1 | 2 | 3 | 4 | 5 | 1 | 2 | 3 | 4 | 5 |
| Acrylic emulsion*1 | Acrylic base resin | 60 | 60 | 60 | 60 | | | 60 | 60 | | |
| Acrylic emulsion*2 | emulsion | | | | | | | | | 60 | |
| Acrylic emulsion*3 | | | | | | | 60 | | | | |
| Acrylic emulsion*4 | | | | | | 60 | | | | | |
| Acrylic base resin*5 | Acrylic base resin | | | | | | | | | | 10 |
| Diethylene glycol | Film-forming aid | 10 | 10 | 10 | | 10 | 10 | 10 | 10 | 10 | 10 |
| diethyl ether | | | | | | | | | | | |
| Dipropylene glycol | | | | | 10 | | | | | | |
| monobutyl ether | | | | | | | | | | | |
| Aminomethylpropanol | pH modifier | 2.25 | 1.5 | 3 | 1.5 | 3 | 1.5 | | | 1.5 | 3 |
| 10% aqueous ammonia | | 1.5 | 3 | | 3 | 1.5 | 3 | 6 | | 3 | |
| Citric acid | *7 | | | | | | | | | | 1 |
| Red 226 dispersion | Colorant | 1 | 1 | 1 | 1 | 1 | 1 | 1 | 1 | 1 | 1 |
| 3% Lucentite SWN*6 | Thickener | | | | | | | | | 10 | |
| Phenoxyethanol | Fungicide | 0.8 | 0.8 | 0.8 | 0.8 | 0.8 | 0.8 | 0.8 | 0.8 | 0.8 | 0.8 |
| Refined water | Solvent | 24.45 | 23.7 | 25.2 | 23.7 | 23.7 | 23.7 | 22.2 | 28.2 | 13.7 | 74.2 |
| pH of composition | | 7.1 | 7 | 7.1 | 7 | 7.5 | 7 | 7 | 4.5 | 7 | 7.3 |
| Hardness | | ◯ | ◯ | ◯ | ◯ | ◯ | X | ◯ | ◯ | ◯ | ◯ |
| Cool water resistance | | ◯ | ◯ | Δ | ◯ | ◯ | ◯ | ◯ | ◯ | ◯ | X |
| Removability | | ◯ | ◯ | ◯ | ◯ | Δ | ◯ | X | X | X | X |

*1solid content: 40%, acid value: 60 mg KOH/g, Tg: 60° C., alkali-soluble
*2solid content: 40%, acid value: 33 mg KOH/g, Tg: 60° C., alkali-soluble
*3solid content: 40%, acid value: 60 mg KOH/g, Tg: 40° C., alkali-soluble
*4solid content: 40%, acid value: 160 mg KOH/g, Tg: 60° C., alkali-soluble
*5AMPHOMER HC: alkali-soluble resin
*6synthetic smectite manufactured by Co-op Chemical Co., Ltd.
*7: added to modify pH as the control example

As apparent from the results shown in Table 1 described above, it has been found that the manicure compositions prepared in Examples 1 to 5 falling in the scope of the present invention are excellent in a hardness, a cold water resistance and removability as compared with the manicure compositions prepared in Comparative Examples 1 to 5 falling outside the scope of the present invention. It has been confirmed that the above situation provides the removing type aqueous manicure composition which can readily be removed by hot water and soaps without using conventional removers and therefore does not damage nails and which does not cause troubles in a daily life since stable to cold water and can be used easily by children and aged persons.

Provided is a removing type aqueous manicure composition which is mild to nails while staying thereon and which can readily be removed by hot water and commercial soaps without using removers containing acetone and acetate esters, so that it can meet a volatile organic compound (VOC) emission control.

1. A removing type aqueous manicure composition comprising at least water, an emulsion of an acrylic base resin which has a glass transition temperature (Tg) of 50° C. or higher and which is alkali-soluble, a film-forming aid, and a pH modifier of non-volatile at ambient temperature.

2. The removing type aqueous manicure composition as described in claim 1, further comprising a pH modifier of volatile at ambient temperature.

3. The removing type aqueous manicure composition as described in claim 1, comprising the emulsion of the alkali-soluble acrylic base resin having an acid value of 40 mg KOH/g or more.
